(19) United States
(12) Patent Application Publication
(10) Pub. No.: US 2003/0087312 A1
(43) Pub. Date: May 8, 2003

(54) Detection of estrogen receptor-beta and methods for diagnosis and classification of cancer

(76) Inventors: Robert J. Christy, San Antonio, TX (US); Yi-Chun J. Wang, San Antonio, TX (US); Suzanne A.W. Fuqua, Sugar Land, TX (US)

Correspondence Address: C. Steven McDaniel, Esq., McDaniel & Associates, P.C., P.O. Box 2244, Austin, TX, 78768-2244, US

(21) Appl. No.: 09/879,746

(22) Filed: Jun. 11, 2001

Related U.S. Application Data
(60) Provisional application No. 60/211,234, filed on Jun. 12, 2000.

(51) Int. Cl. G01N 33/53; G01N 33/567; G01N 33/537; G01N 33/543; C12N 5/06; C07K 16/28

(52) U.S. Cl. 435/7.2; 435/334; 435/7.92; 530/388.22; 435/7.5

(57) ABSTRACT

Compositions and methods for detection of estrogen receptor-β in human tissues are provided. The compositions are monoclonal antibodies specific for estrogen receptor-β binding. The method provided is a prognostic method and/or a predictive assay for cancer, including breast cancer. The assay provided uses a estrogen receptor-β antibody that has both high affinity and specificity and can be used in archival tissue sections.

### BACKGROUND OF THE INVENTION

The effects of estrogens in biological systems are varied. The effects of these hormones are mediated by a family of receptors known as the estrogen receptors that include estrogen receptor-α (ERA) and estrogen receptor-β (ERB). The estrogen receptor was first cloned in 1986 and at that time only one estrogen receptor had been identified, now known as ERA. Recently, the second receptor isoform, ERB, was discovered in rat prostate and ovary tissue (Kuiper, G G J M et al. 1996. Proc. Natl. Acad. Sci. USA 93:5925-5930). This second isoform estrogen receptor has now been cloned in human and mouse, with the human gene for ERB mapped to chromosome 14q22-24 (Enmark, E. et al. 1997. J Clin. Endocrinol. Metab. 82:4258-4265). The ERB protein is a 54.2 kDa protein in humans (Chang, W. Y. and G. S. Prins. 1999. The Prostate 40:115-124).

ERA is a useful clinical biomarker of breast cancer progression and is routinely monitored by clinicians in order to estimate patient prognosis and select optimal therapies. In guidelines published by the ASCO Tumor Marker Expert Panel (Ravdin, P. M. 1997. Prognostic factors in Breast Cancer, American Society of Clinical Oncology Educational Book, p. 217-227), the estrogen receptor (i.e., ERA) and the progesterone receptor were the only biomarkers recommended for routine use in management of patients with breast cancer. The estrogen receptor assay is most useful as a tool to aid in selection of therapies for breast cancer. Patients having an estrogen-receptor negative tumor seldom respond to endocrine therapy.

Predicting the probability of response to endocrine therapy in estrogen-receptor-positive patients is more difficult. Overall, approximately 50-60% of women with estrogen-receptor-positive breast cancer will receive some degree of benefit from endocrine treatment with the anti-estrogen tamoxifen (1992. Lancet 339:1-15). Unfortunately, many patients who initially respond to tamoxifen will eventually recur with estrogen hormone-resistant disease.

Both ERA and ERB are part of a superfamily of nuclear hormone receptors that function as transcription factors when they are bound to their respective ligands. Nuclear receptors share common structural and functional features. ERA contains 595 amino acids with a central DNA binding domain (DBD) along with a carboxy-terminal hormone binding domain (HBD). ERB is somewhat shorter than ERA and is predicted to contain 530 amino acids (Ogawa, S. et al. 1998. Biochem. Biophys. Res. Commun. 243:122-126). The region of highest homology between ERA and ERB is in the DBD (97%), with much less homology in the HBD (54%). There is even less conservation in the amino-terminal activation function-1 domain (AF-1). Since estrogen-receptor-mediated gene transcription is stimulated through the two transactivation domains, AF-1 and the carboxy-terminal AF-2 domain (Kumar, V. et al. 1986. EMBO J. 5:2231-2236; Kumar, V. et al. 1987. Cell 51:941-951; Tora, L. et al. 1989. Cell 59:477-487), ERB may regulate different genes from ERA and molecular mechanisms regulating ERB activity are likely distinct from those regulating ERA activity.

In fact, studies have shown that ERB has negligible autonomous AF-1 activity as compared to ERA when estrogen response element-based reporter activity is examined (Cowley, S. M. and M. G. Parker 1999. J. Steroid Biochem. Mol. Biol. 69:165-175; Hall, J. M. and D. P. McDonnell. 1999. Endocrinology 140:5566-5578), a result that explains the lack of agonist activity exhibited by tamoxifen with ERB on artificial estrogen response element-based reporter systems. Further, the three dimensional structure of the ERB HBD indicates that its AF-2 co-activator contact surface is similar but not identical to ERA (Pike, A. C. et al. 1999. EMBO J. 18:4608-4618). These data suggest that there is a differential recruitment of cellular factors, such as estrogen receptor co-regulatory proteins, to the transcription complex or preferential binding of some co-regulatory proteins to ERB when ERB is expressed.

The differences in ERA and ERB in the ligand binding domain have led to examination of the ligand-binding profiles of ERB versus ERA. Studies have shown that these two receptors share similar profiles in terms of specificity and affinity for compounds such as estrial, estrone, estradiol-17β, as well as many other known estrogen receptor ligands. However, significant differences in ERB and ERA binding and activation have been identified. The selective estrogen receptor modulator raloxifene binds ERB with an affinity four times lower than ERA (Kuiper, G G J M et al. 1998. Endocrinology 13:4252-4263). Regulation of ERB-mediated gene transcription at AP1 enhancer elements has been shown to be different than regulation of ERA (Paech, K. et al. 1997. Science 277:1508-1510). Also, unlike ERA, ERB transcriptional activity is inactive with estradiol (Paech, K. et al. 1997. Science 277:1508-1510). These differences in ERB binding and transcriptional activity may be related to selective activity of the receptor in different tissues. ERB has been identified in prostate tumor tissue, testis, ovary, thymus, spleen, breast, adipose tissue, uterus, pituitary adenomas, and kidney.

The physiological function of ERB has not been fully elucidated. Using ERB knockout mice, it has been shown that ERB is important for growth control of the epithelium of the urogenital tract and that ERB expression may afford protection against hyperproliferation and carcinogenesis (Gustafsson, J. 1999. J. Endocrinol. 163:379-383). These results in animals are supported by clinical findings (Leygue, E. et al. 1998. Cancer Res. 58:3197-3201; Pujol, P. et al. 1998. Cancer Res. 58:5367-5373).

In some circumstances and under certain conditions, ERB interferes with the estrogen responses elicited by stimulation of ERA (Hall, J. M. and D. P. McDonald. 1999. Endocrinology 140:5566-5578). It has been found that ERB expression can inhibit tamoxifen's agonistic effects on ERA in breast cancer cells. Further, a study of 40 tumor samples showed that ERB expression was significantly lower in progesterone receptor-positive tumors, indicating a role for ERB expression in tumor prognosis determinations since progesterone receptor is a biomarker and predictor of response to tamoxifen therapy (Dotzlaw, H. et al. 1999. Cancer Res. 59:529-532).

What is needed are methods and materials to independently assess both the ERA and ERB status of breast tumor cells in order to define more specifically the clinical outcome of a patient. ERB expression may correlate with a poor prognosis for response to tamoxifen therapy and would provide a valuable tool in determining the appropriate therapy for breast cancer patients. In fact, the difference in expression of ERA versus ERB has been suggested to be important in the progression of breast cancer (Fuqua, S. A. W. et al. 1999. Cancer Res. 59:5425-5428).

Antibodies, both monoclonal and polyclonal, have been developed against the estrogen receptor that are not selective for ERA versus ERB. Traish (U.S. Pat. No. 5,744,356) describes a monoclonal antibody that is specific for an epitope within residues 1-184 of an estrogen receptor protein that is useful for detection of the activated 4S and 5S forms of the estrogen receptor, distinguishing between the hormone-activated form of the receptor and the native or unactivated form of the receptor. In addition, monoclonal and polyclonal antibodies against ERA are available commercially (Novocastra Laboratories Ltd., Newcastle upon Tyne, England). Antibodies developed to date have not solved the need for independent methods and materials allowing simultaneous detection of the two receptors in a tissue.

Polyclonal antibodies against ERB are commercially available (Alpha Diagnostic International, Inc., San Antonio, Tex.). These commercially available polyclonal antibodies are directed to the carboxy terminus of the ERB receptor protein.

Fuqua et al. (1999. Cancer Res. 59:5425-5428) reported development of ERB-specific antibodies for characterization of ERB protein expression in breast cancer cell lines and tumors using Western Blot analysis. In this report, monoclonal antibodies were made to a peptide representing the first 18 amino acids (N-terminal region) of the longest ERB open reading frames reported (amino acids 39-146 of the ERB protein) while polyclonal antibodies were made against a peptide within the ERB B domain. To date, all of the ERB antibodies reported can only be used to detect the presence of ERB in frozen tissue samples; none of the antibodies, either polyclonal or monoclonal, bind to paraffin-embedded, fixed tissue samples.

Since most pathological samples are fixed and embedded in paraffin for storage, there is a need for methods of ERB detection that are specific for the beta form of the estrogen receptor and that can be used in immunocytochemical studies of paraffin-embedded fixed tissue sections. In order to better understand the role of ERB in tissues, methods and materials for detection that are specific for this receptor over ERA are needed that are useful in fixed histological specimens.

Background For Extension of Usefulness of Anti-ER-Beta Antibodies

Nuclear Receptors

Nuclear receptors are ligand activated transcription factors that have been shown to play critical roles in development, cell growth, cell differentiation and homeostasis (Mangelsdorf, D. F. et al. 1995. Cell 83:835-839). The nuclear receptor superfamily can be subdivided into steroid receptors (estrogen, progesterone, etc.,) and nonsteroid receptors thyroid, retinoic acid, etc.) (Beato, M. et al. 1995. Cell 83:851-857; Mangelsdorf, D. et al. 1995. Cell 83: 841-850). All nuclear receptors have similar, but not identical, structure which can be divided into several functional domains (Mangelsdorf, D. et al. 1995. Cell 83: 841-850). This includes a DNA binding domain which is highly conserved among all the nuclear receptors and two transcriptional activation domains. The transcriptional activation domain near the N-terminal is constitutively active and is known as AF-1 (activation function 1). The second transcriptional activation domain, located in the C-terminal region, is much stronger and hormonally regulated. It is known as the AF-2 (activation function 2). The AF-2 domain has unique conformations as an aporeceptor, agonist bound receptor or antagonist bound receptor. Unliganded steroid receptors are complexed with chaperone proteins such as the heat shock proteins and remain inactive (Smith, D. F. et al. 1992. J Biol Chem 267:1350-1356). Upon binding of ligand, the receptor forms homodimers and binds to DNA. In contrast, nonsteroid receptors are unliganded heterodimers, already bound to DNA and are complexed with corepressor complexes which inhibit gene transcription (Magelsdorf, D. et al. 1992. J Biol Chem 267:1350-1356). Upon the addition of ligand, the corepressor nuclear receptor complex is destabilized and transcription is activated. These changes in conformation when agonists bind allow other proteins, coactivators, to interact with the receptor to produce an activated transcription factor. The exact composition of these nuclear receptor coactivator/corepressor complexes in the cell is not known, but a large number of proteins have been shown to interact with nuclear receptors on the basis of direct biochemical interaction both in vivo and in vitro and have been termed coactivators, corepressors and integrators (Darimont, B. D. et al. 1998. Genes & Dev 12:3343-3356; Wong, C. et al. 1998. Mo & Cell Biol 18:5724-5733; Horwitz, K. B. et al. 1996. Mol Endo 10:1167-1177). These factors add another level of complexity in the mechanism of transactivation of nuclear hormone receptors with the amounts and combinations of these cofactors being extremely important in the regulation of the receptor activity.

Estrogen Receptor Hetero Dimers and Isoforms

The ER-α and ER-β proteins have been shown to form homodimers and bind DNA with equal affinity (Ogawa, S. et al. 1998. Biochem. Biophys. Res. Comm. 243:122-126; Petterson, K. et al. 1997. Mol. Endo. 11:1486-1496). In addition experiments have shown that ER-α and ER-β heterodimers can form both in vitro and in intact cells. This suggests that two new estrogen signaling pathways exist in cells that exclusively express ER-β via homodimer formation in cells that exclusively express one receptor subtype and homo-and hetero-dimer formation in cells that express both receptor subtypes. With the findings that levels of both estrogen receptors and isoforms are altered in tumors leads to the possibility that the change in protein levels alter the potential ER dimers that may form and their activity (Leygue, E. et al. 1999. Cancer Research 59:1175-1179). Indeed, ER-βcx is truncated at the C-terminal end, but contains an extra 26 amino acids due to an alternative splicing and it no longer binds ligand. But, ER-βcx can form heterodimers with ER-α and therefore inhibit DNA binding and ER-α mediated estrogen transcription by acting as a dominant negative receptor (Ogawa, S. et al. 1998. Nuc. Acids Res. 26:3505-3512).

Similar to other nuclear receptors both estrogen receptors have been shown to have additional isoforms generated by alternative splicing (Murphy, L. C. et al. 1997. Ann. Medicine 29:221-224; Shupnick, M. A. et al. 1998. Mol. Cell. Endo. 138:199-203; Moore, J. T. et al. 1998. Biochem BioPhys. Res. Comm. 247:75-75). These alternative splice variants again add an additional level of complexity to understanding estrogen action and the biological properties of a tumor's response to estrogen and antiestrogen treatment (Shupnick, M. A. et al. 1998. Mol. Cell. Endo. 138:199-203). This includes five isoforms that have been reported at the mRNA level for ER-β (FIG. 4)(Moore, J. T. et al. 1998. Biochem BioPhys. Res. Comm. 247:75-75). The figure indicates the carboxyl terminal end of the ER-β protein and indicates alterations in primary sequences due to alternative splicing of the mRNA. This variation in amino acid sequence will be used to generate synthetic peptides for antibody production.

Estrogen Receptors in Cancer

The cloning of the ER-β receptor has prompted investigators to reevaluate the molecular basis for estrogen action and the role of estrogen receptors in tumors from tissues where ER-β is normally expressed and in breast cancer (Speirs, V. et al. Cancer Res. 59:525-528). Most studies to date have had to use RT-PCR to measure mRNA levels rather than immunohistochemistry (IHC) to determine ER-β protein levels because of lack of ER-β specific antibodies that work in IHC (Hanstein, B. et al. 1999. Mol. Endo. 13:129-137). Therefore, all these results are tempered by the fact that it is not known if there is a direct correlation between the levels of mRNA and the level of protein for ER-β in normal and/or tumor tissue. Full length ER-β has been shown to be expressed in normal breast tissue and human breast tumors (Dotzlaw, H. et al. 1997. Endocrin Meta. 82:2371-2374). The ratio of ER-α and ER-β change during tumor grade suggesting that both receptor types have a role in tumor formation (Leygue, E. et al. 1998. Cancer Res. 58:3197-3201). Recently a PCR assay was developed using triple primer sets for the co-amplification of ER-β and several isoforms. The results indicate that at the mRNA level changes in the relative expression of the fall length and several isoforms occurs during breast tumorigenesis and cancer progression (Leygue, E. et al. 1999. Cancer Research 59:1175-1179; Leygue, E. et al. 1998. Cancer Res. 58:3197-3201).

Very recently two studies have been done to compliment the RNA studies previously reported. In the first study by Fuqua et al., they have shown at the protein level that ER-β and several isoforms are expressed in breast cancer cell lines and tumors and correlated this to RNA levels (Fuqua, S. A. W. et al. 1999. Cancer Res. 59:4525-4528). In the second study by Jarvinen, et al. (Jarvinen, T. A. H. et al. 2000. Am J Pathology 156:29-35) they used a polyclonal antibody (PA1313, Affinity Bioreagents, Golden, Colo.) on frozen sections to analyze ER-β expression by immunohistochemistry (IHC). Their results indicate that ER-β is expressed in normal epithelial cells and in 60% of breast tumors and ER-β is often coexpressed with ER-α and PR. They conclude that ER-β can be used along with ER-α and PR to determine if a tumor will respond to hormone therapy. It is important to note in this study that the tumor sections were frozen and their attempts to use paraffin-embedded tissue (the way most clinical samples are stored) did not work (Jarvinen, T. A. H. et al. 2000. Am J Pathology 156:29-35).

Estrogen Receptor in Hormone Therapy

The mRNA level of ER-β accessed by PCR in tumors showed that ER-β levels were much lower in progesterone receptor positive (PR+) tumors as compared to PR− tumors and ER-β levels can be reduced by progestin in breast cancer cells (Dotzlaw, H. et al. 1999. Cancer Res. 49:529-532). In comparing ligand binding of ER-α and ER-β, both bind physiological ligands and some antagonists with equal affinity, but differ in there ability to bind and affect gene transcription in response to other specific antiestrogens SERMs and environmental estrogens (Tremblay, A. et al. 1998. Endo 139:111-118; Giguere, V. et al. 1998. Steroids 63:335-339, 1998). ER-β binds to estrogen metabolites with similar affinity as ER-α, including estrogen, diethylstilbesterol, estriol and estrone. Binding of estrogen antagonists such as raloxifene and tamoxifen were also identical (Kuiper, G. G. J. M. m et al. 1997. Endocrinology 138:863-870). A difference in binding affinity does occur in that ER-β binds xenoestrogens and phytoestrogens (genistein) significantly better than ER-α (Kuiper, G. G. J. M. et al. 1998. Endocrinology 13:4252-4263) indicating that ER-β plays a unique role in the physiologic functions of natural estrogens.

Selective estrogen receptor modulators (SERMs) have been shown to act as either ER antagonists or agonist depending on the type of tissue analyzed and the dose administered. An example is the new SERM raloxifene which acts as an antiestrogen in breast tumor tissue and brain, but it maintains estrogen-like effects in bone and heart and has actually shown an increase in bone density, but does not stimulate endometrial growth or increase the chance of breast cancer (Raafat, A. M. et al. 1998. Endo. 140:2570-2580). This is similar to tamoxifen except that tamoxifen is an antagonist in brain tissue and also exerts estrogenic activity in endometrium to increase the cancer risk. The complexity of the problem is also effected in tissues where both receptors are expressed with the varying ratios of ER-α and ER-β and their isoforms may result in changing ratios of ER-α and ER-β homo-and hetero-dimers. These experiments along with the studies described above raised the possibility to the present inventors that ER-β is potentially a useful marker in determining hormone therapy responsiveness in some breast tumors. This inverse relationship between PR, which is a good prognostic variable and marker of response to hormone therapy may suggest that ER-β levels or increases in its expression may correlate with a poorer prognosis (Dotzlaw, H. et al. 1999. Cancer Res 59:529-532). Again these studies results need to be confirmed at the protein level.

Prognostic and Predictive Indicators of Cancer

Morality from breast cancer has declined this decade due to earlier diagnosis and the widespread use of adjuvant chemotherapy and hormonal therapy after surgery. About 60% of patients with advanced breast cancer respond to chemotherapy with partial tumor regression, but only 15-20% have complete tumor regression. In early breast cancer, about 50% of those patients estimated to have a poor prognosis are now treated with several months of adjuvant chemotherapy. Recurrence and mortality are reduced only by 20-40% in such patients. Thus, many patients treated with toxic drugs do not stand to benefit because of tumor drug resistance, while others are not offered chemotherapy because they are mistakenly thought to have a good prognosis. Identifying the unknown changes or specific genetic alterations in an individual patient's tumor that correlate with tumor aggressiveness and/or response to treatment would have profound clinical implications.

Prognostic markers are those molecular features that correlate with the natural aggressiveness of a tumor, and predictive markers are those that correlate with treatment response. The estrogen receptor protein is a good example of both: ER+ tumors are more indolent, and they are also much more responsive to hormonal treatments such as tamoxifen. Overexpression of the HER-2/neu oncogene correlates with more rapid tumor recurrence, but also with a better response to doxyrubicin-based chemotherapy (Muss, H. B. et al. 1994.N. Engl. J. Med. 330:1260-1266; Seshadri, R. et al. 1993. Clin. Oncol., 11:1936-1942; Slamon, D. J. et al. 1989. Science 244:707-12).

Breast cancer is a very common disease that afflicts millions of women worldwide (Slamon, D. J. et al. 1989. Science 244:707-12; Eisinger, F. et al. 1998. Annals of Oncology 9:939-950). Unfortunately, the clinical course of primary breast cancer varies considerably from patient to patient. Some patients have very long disease-free survival, while others experience a rapid deterioration with early recurrence of their breast cancer, followed shortly by death. Due to the complexity and heterogeneity of these tumors, determining the proper treatment presents several dilemmas for the physician and patient. Among these is the question of whether or not to use systemic adjuvant therapy following surgery. And, if so which therapy—hormonal, chemotherapy, or both—is best for a particular patient? In order to choose the best treatment for a specific patient, a physician must firsts determine the likelihood that the patient will have a recurrence of disease if no systemic therapy is administered after surgery.

Prognostic data is the key to this decision. Some of the disease variability is undoubtedly explained by differences in tumor growth rates, invasiveness, metastatic potential and other mechanisms that we do not fully understand. It is imperative that we develop biomarkers that measure these characteristics, either directly or indirectly, so those individual patients can be classified into subsets with varying risk of disease recurrence.

Three clinical situations for which prognostic factors would be useful have been described previously (Elledge, R. M. et al. 1994. Cancer Research 54:3752-3757). The first involves identifying patients whose prognosis is so good following surgery alone that the benefits of systemic adjuvant therapy would not out-weigh the toxic effects. Approximately 70% of node-negative patients and 25% of node-positive patients remain alive for at least 10 years after surgery without any other therapy (Hilsenbeck, S. G. et al. 1992. Human Pathology 23:601-602; Hilsenbeck, S. G. et al. 1992. Breast Cancer Res. Treat. 22:197-206). The issue is whether subsets of patients with extremely low risk of disease recurrence can be identified so that chemotherapy can be avoided.

The second situation involves identifying patients whose prognosis is so poor even with conventional chemotherapy treatment that more aggressive forms of therapy might be warranted. In spite of improvements in adjuvant therapies, there are some patients whose cancers rapidly recur following conventional adjuvant therapy. These patients might be candidates for more aggressive regimens such as high-does chemotherapy followed by bone marrow or peripheral stem cell rescue. Our collaborators have already begun to address this problem by creating models to identify such patients.

The third, and perhaps most important, is the use of prognostic markers to predict which patients are or are not likely to benefit from specific therapies. While it is useful to accurately predict the likely clinical outcome of a patient's breast cancer, it is even more important to be able to alter the natural history of disease and to prevent or delay its recurrence. Therefore, biomarkers that are predictive of the utility of specific therapies, including specific chemotherapeutic agents, would be extremely useful in the clinic.

Thus, it is critical that monoclonal antibodies, and the hybridomas that produce them, that specifically bind estrogen receptor-β protein by located and characterized. In particular, such antibodies should specifically bind to an epitope within amino acid residues 1-146 or to an epitope within amino acid residues 1-36 of the estrogen receptor-β protein, and even more particularly that such antibodies effectively bind to the receptors in fixed paraffin-embedded tissue samples. With such reagents, it would be desirable to build kits for the detection of receptors protein in a biological sample. It would also be highly advantageous to utilize ER-β as a marker in determining hormone therapy responsiveness in breast tumors. It would also be a major advancement if reagents, tests and kits were feasible wherein, depending on the level of expression, each ER subtype could be evaluated for its impact on the ability of a specific tumor type to respond to clinically important anticancer agents. It might be possible, using such invention to determine the protein distribution and function of ER-β and its isoforms in various estrogen responsive tissues. If so, such inventions would be critical to predicting pharmacologic and physiologic impact as well as to facilitate the development of tissue specific estrogen agonists and antagonists to treat the disease in only the affected tissue. Additionally, using such inventions, it might be possible to understand the mechanisms of action of both ER types and to help develop superior SERMs than those currently available (tamoxifen, raloxifene). It would be especially valuable to be able to block estrogen action in a specific tissue where it promotes growth and possibly cancer (breast) but does not effect estrogen action on bone and brain where it protects women from osteoporosis and Alzheimers disease, respectively. These inventions will be crucial in addressing the challenge to eliminate the adverse effects of current selective estrogen receptor modulators (SERMs) and to minimize the problems with hormone replacement therapy.

### SUMMARY OF THE INVENTION

The present invention is directed to a monoclonal antibody that specifically binds estrogen receptor-β protein.

One aspect of the invention is a monoclonal antibody that specifically binds to an epitope within amino acid residues 1-146 of the estrogen receptor-β protein in fixed paraffin-embedded tissue samples.

Another aspect of the invention is a monoclonal antibody that specifically binds to an epitope within amino acid residues 1-36 of the protein. A preferred embodiment of the monoclonal antibody being an anti-estrogen receptor-β produced by hybridoma clone 14C8.

Yet another aspect of the invention is a hybridoma that secretes a monoclonal antibody that specifically binds to an epitope within amino acid residues 1-146 of the estrogen receptors protein in fixed paraffin-embedded tissue samples.

Still yet another aspect of the invention is a kit for the detection of receptor-β protein in a biological sample. Still another aspect of the invention is the use of ER-β as a useful marker in determining hormone therapy responsiveness in breast tumors.

Still another aspect of the present invention is the determination of the level of expression for each ER subtype and the impact thereon on the ability of a specific tumor type to respond to clinically important anticancer agents. Use of the reagents, tests and kits of the present invention to determine the protein distribution and function of ER-β and its isoforms in various estrogen responsive tissues is yet another aspect of the invention that allows predicting pharmacologic and physiologic impact of a therapy, as well as to facilitate the development of tissue specific estrogen agonists and antagonists to treat the disease in only the affected tissue.

### DESCRIPTION OF THE DRAWINGS

FIG. 1 depicts a schematic protein map of the homologous regions of ERA and ERB and indicates the regions of ERB targeted for monoclonal antibody development.

FIG. 2 illustrates the binding of monoclonal antibodies directed against ERA and ERB to recombinant ERB.

FIG. 3 illustrates the binding of monoclonal antibodies directed against ERA and ERB to recombinant ERA.

FIG. 4 illustrates the comparison of the deduced carboxyl terminal amino acid sequences of ER-β and specific isoforms that have been isolated from tumor cell lines and tissue.

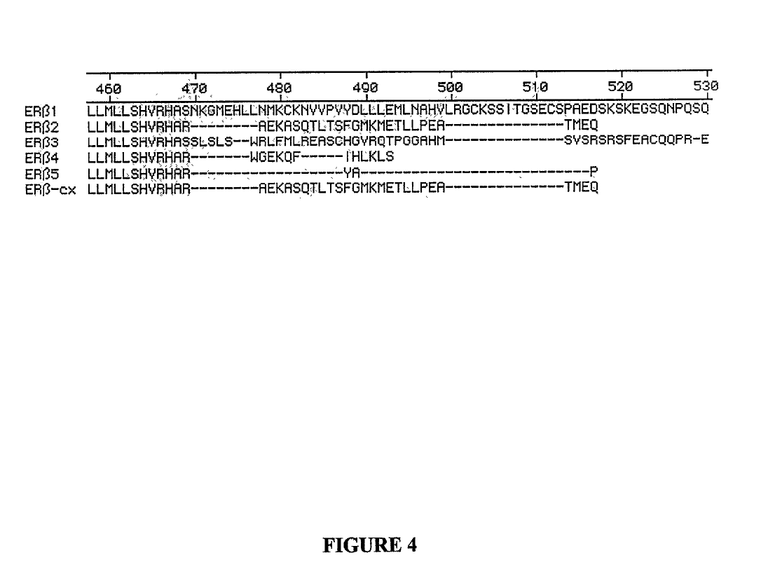

FIG. 5 illustrates immunocytochemical analysis of paraffin-embedded, fixed tissue processed with 14C8 ER-β monoclonal antibody. A. Endocervical epithelium. B. Infiltrating lobular carcinoma of the breast.

### DETAILED DESCRIPTION OF THE INVENTION

ERB-specific monoclonal antibodies have been developed that, unlike previous antibodies against ERB, have both high affinity and specificity for ERB and can be used to detect the presence of ERB in paraffin-embedded fixed (permanent) tissue sections. Such permanent tissue section samples are the type of tissue sections that are archived. Therefore, the present invention provides a valuable clinical tool for examining the role of ERB in tumor progression in archival sections from prospective clinical studies, where “archival” tissue sections are defined as sections that have been fixed and/or paraffin-embedded.

Specific antibodies to ERA are commercially available. Such ERA antibodies, when used with the ERB specific antibodies of the present invention provide a basis for a method of detection of ERB in tissues that express both ERA and ERB and allow for quantification of the levels of expression of ERB as compared to ERA. Studies have suggested that ERB expression indicates a poor prognosis to hormone therapy for certain cancers (the opposite of ERA expression). Therefore, the identification of the expression ratio of ERB/ERA in tissues is useful in the diagnosis and classification of diseases associated with estrogen receptor expression, such as breast cancer.

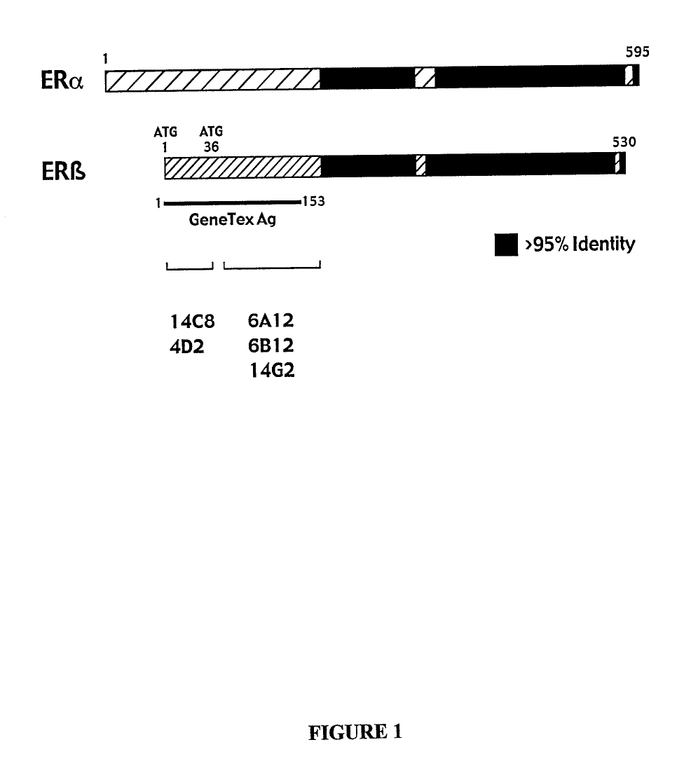

With the recent identification of several variants of ERB in human tissues, five at this time (Moore, J. T. et al. 1998. Biochem. Biophys. Res. Commun. 247:75-78; Leygue, E. et al. 1999. Cancer Res. 59:1175-1179), it has become clear that choosing the correct target region for monoclonal antibody development is critical. This is because the various isoforms are formed by shortening of the carboxy terminal region of the ERB protein. Therefore, antibodies directed to the carboxy terminal region probably recognize only certain isoforms of the ERB protein, rather than the total levels of ERB. In addition, as illustrated in FIG. 1, the amino acid sequence of the carboxy ends of ERA and ERB are greater than 95% homologous, while the amino acid sequence of the amino ends of ERA and ERB are much less homologous.

The inventors reasoned that an antibody that is directed to the N-terminal region of the ERB protein may be more specific for ERB and would recognize all known ERB variants to provide a more accurate assessment of total ERB levels. But, it was not known if antibodies to this region would be successful in histological samples. In the present invention, the N-terminal region of the ERB protein was targeted for monoclonal antibody development.

The resultant antibodies provide an assessment of total ERB binding in a tissue section or biological sample. Several monoclonal antibodies, specific for the amino end of ERB, have been developed and the clones are known as 14C8, 4D2, 6A12, 6B12, and 14G2. These antibodies are directed to the first 146 amino acids of the ERB protein (FIG. 1). For the monoclonal antibodies 14C8 and 4D2, the epitopes are located within the first 36 amino acids. For clones 6A12, 6B12 and 14G2, the epitopes are located within amino acids 36-146. The production of these monoclonal antibodies is set forth in more detail in Example 1.

The various monoclonal antibodies developed have been tested for their affinity and specificity for binding ERB in both ELISAs (see Example 2), western blots (see Example 3) and immunocytochemical techniques (see Example 4). These methods are standard screening methods used to characterize the specificity and affinity of antibody binding.

The present invention includes both compositions and methods for detecting ERB in biological samples. In the context of the invention, “biological samples” would include but not be limited to tissue samples taken from a patient for the purpose of pathology examination. These samples could be tumor tissue samples as well as non-tumor tissue samples, and can be fresh samples as well as archival samples. Other types of biological samples could be tested for the presence of ERB using the compositions and methods of the present invention and would include fixed cell cultures or cell preparations, as well as gels run from samples prepared from tissue or cell samples.

The monoclonal antibodies of the present invention can be used for the detection of ERB in biological samples using known methods for detection of antibody binding. Such methods, include but are not be limited to, radioimmunoassays, immunoprecipitation, western blotting, enzyme-linked immunosorbent assays (ELISA), and immunohistochemistry or immunocytochemistry. Example 5 describes the use of these monoclonals in immunocytochemical kits for the analysis of tissue samples.

The following non-limiting examples are provided to better illustrate the claimed invention.

### EXAMPLES

### Example 1

### Monoclonal Antibody Preparation

The antigen used for production of monoclonal antibodies was expressed from a cDNA fragment from the 5′-end of ERB and was made from total RNA extracted from the human breast cancer cell line MCF-7. The 5′ PCR primer was GTXERβ-F1: 5′-ctggatcc ATG GAT ATA AAA AAC TCA CCA TC -3′ (NT#410-441 from GenBank Sequence AF51427) and the 3′ PCR primer was ERY-5(RC) Sa1 I 5′gcgtcgac TGA GCA TCC CTC TTT GAA CCT GGA C -3′ (NT# 833-864 from GenBank Sequence AF51427) (upper case letters denote the protein coding sequence while lower case letters denote the untranslated sequence). For cloning and expression of the antigen, the PCR ERB cDNA fragment was digested with appropriate restriction enzymes and then purified from gels. The purified DNA fragment was cloned into pET28a+ expression vector (Novagen, Madison, Wis.). After screening and characterization of the initial clones, the positive clones were grown to a larger volume and the expressed protein was purified through His-Bind (Pierce, Madison, Wis.) column chromatography under denatured conditions to near homogeneity.

Five female Balb/c mice were immunized with purified recombinant ERB. The primary immunization antigen consisted of 50 μg of purified ERB protein suspended in 150 μl of phosphate-buffered saline (PBS) mixed with an equivalent concentration of Freund's complete adjuvant administered intradermally and intraperitoneally into mice. Booster immunizations contained 50 μg ERB protein and incomplete adjuvant Sera from immunized animals was tested for the presence of immunoreactive antibody by ELISA and western blots against the purified recombinant ERB protein.

Spleens from mice producing immunoreactive antibody were fused to NS-1 myeloma cells using standard techniques. After fusion, cells were grown in HAT selective media. Supernatants from growing hybridoma were tested for ERB-specific antibodies by ELISA and western blotting using purified recombinant ERB protein as antigen. All cultures were examined for antibody secretion during the expansion of the clones.

Clones determined to be positive by ELISA and western blots were single-cell cloned. Hybridoma cells were grown from frozen stocks and were single-cell cloned by serial dilution methods. Clones grown from single cells were screened again by ELISA in order to prevent the possibility of multiple species of monoclonal antibody in the parental hybridoma culture. Cell lines were expanded and a portion was frozen to preserve antibody-producing cell lines. Once a larger volume of overgrown supernatant was obtained, the specific monoclonal antibodies were purified and concentrated to obtain high-purity antibody stock.

### Example 2

### ELISA Analysis

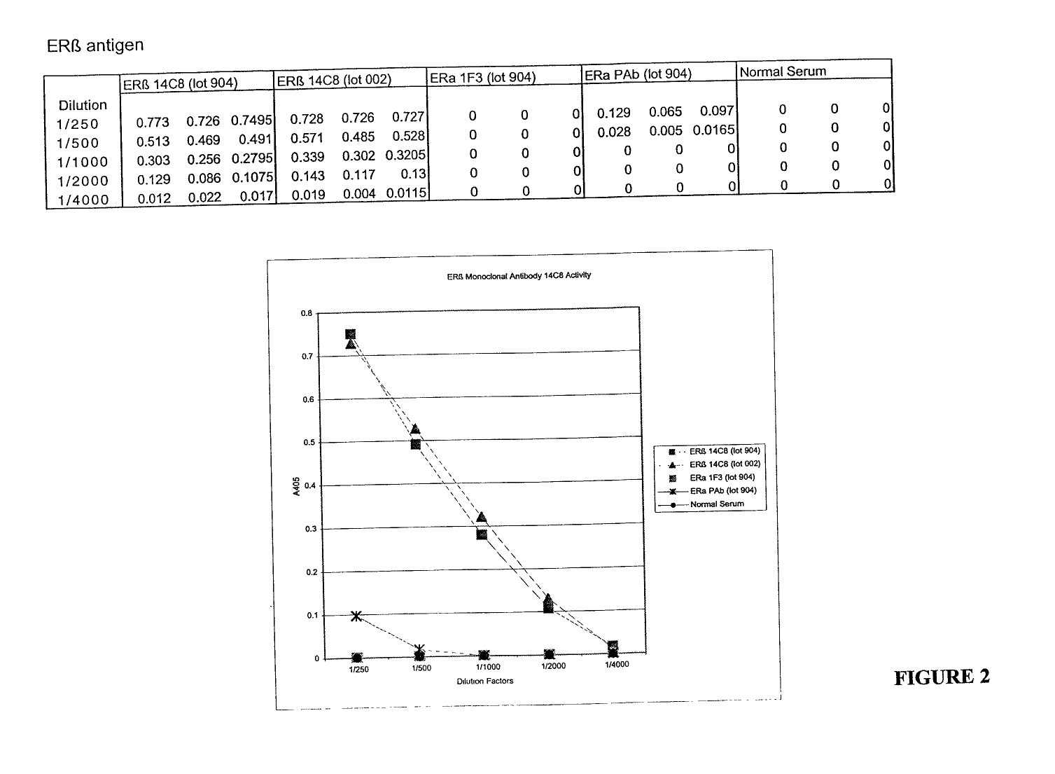

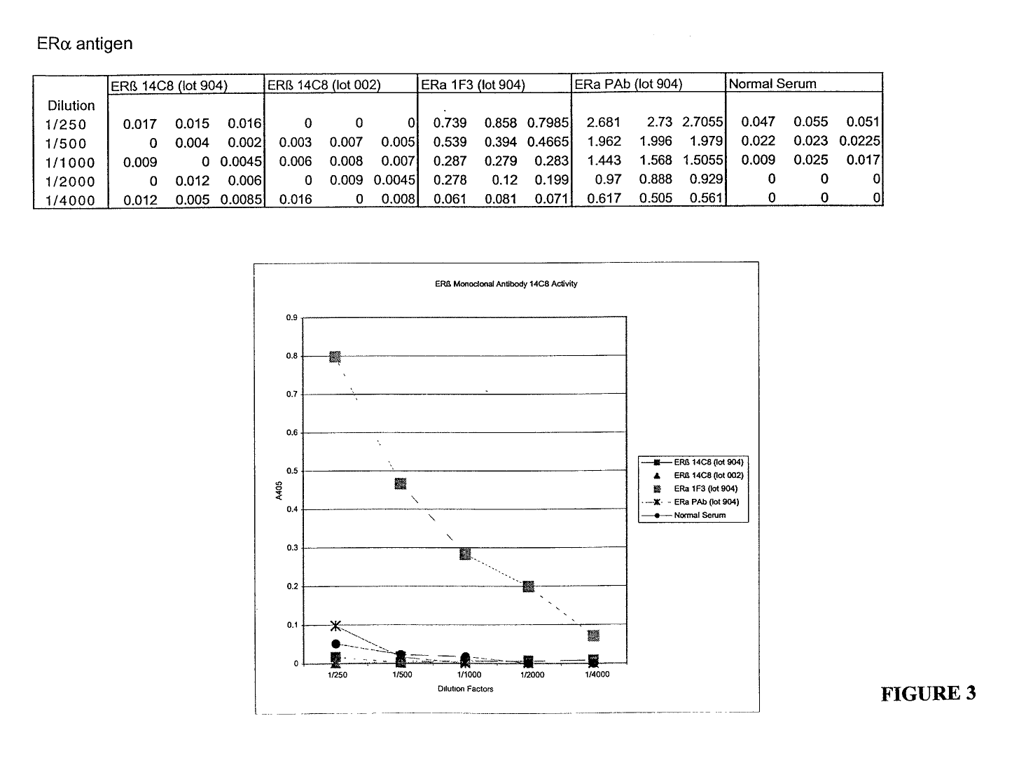

The purified monoclonals were tested for their specificity and affinity. For example, FIG. 2 illustrates the binding of two lots of 14C8 to ERB and FIG. 3 illustrates the binding of the two lots of 14C8 to ERA. The analyses were done by a standard ELISA in 96 well microtiter plates. Each well contained 500 ng of purified ERB or ERA recombinant protein.

Purified anti-ERB 14C8 monoclonal antibody (lots 904 and 002), anti-ERA 1F3 antibody (lot 904), were normalized to a concentration of 1 mg/ml. Various dilutions of these antibodies, ERA polyclonal antibody (lot 904) and normal mouse serum (i.e., 1/250, 1/500, 1/1000, 1/2000 and 1/4000) were made and tested for their binding to recombinant ERA and ERB. Table 1 gives the binding data for these antibodies to both recombinant ERA and ERB in A405 which is proportional to the amount of antibody that bound to the 500 ng of protein in the well.

TABLE 1

| | | | | | |
|---|---|---|---|---|---|
| Dilution | Erβ 14C8 (lot 904) | Erβ 14C8 (lot 002) | Era 1F3 (lot 904) | Era PA b (lot 904) | Normal Serum |

Erβ antigen

| | | | | | | | | | | | | | | | |
|---|---|---|---|---|---|---|---|---|---|---|---|---|---|---|---|
| 1/250 | 0.773 | 0.726 | 0.7495 | 0.728 | 0.726 | 0.727 | 0 | 0 | 0 | 0.129 | 0.065 | 0.097 | 0 | 0 | 0 |
| 1/500 | 0.513 | 0.469 | 0.491 | 0.571 | 0.485 | 0.528 | 0 | 0 | 0 | 0.028 | 0.005 | 0.0165 | 0 | 0 | 0 |
| 1/1000 | 0.303 | 0.248 | 0.2795 | 0.339 | 0.302 | 0.3205 | 0 | 0 | 0 | 0 | 0 | 0 | 0 | 0 | 0 |
| 1/2000 | .0129 | 0.086 | 0.1075 | 0.143 | 0.117 | 0.13 | 0 | 0 | 0 | 0 | 0 | 0 | 0 | 0 | 0 |
| 1/4000 | 0.012 | 0.022 | 0.017 | 0.019 | 0.004 | 0.0115 | 0 | 0 | 0 | 0 | 0 | 0 | 0 | 0 | 0 |

Erα antigen

| | | | | | | | | | | | | | | | |
|---|---|---|---|---|---|---|---|---|---|---|---|---|---|---|---|
| 1/250 | 0.017 | 0.0155 | 0.016 | 0 | 0 | 0 | 0.739 | 0.858 | 0.7985 | 2.881 | 2.73 | 0.097 | 0.047 | 0.055 | 0.051 |
| 1/500 | 0 | 0.004 | 0.002 | 0.003 | 0.007 | 0.005 | 0.539 | 0.394 | 0.4665 | 1.962 | 0.005 | 0.0165 | 0.022 | 0.023 | 0.0225 |
| 1/1000 | 0.009 | 0.248 | 0.0045 | 0.006 | 0.008 | 0.007 | 0.287 | 0.279 | 0.283 | 1.443 | 1.568 | 1.5055 | 0.009 | 0.025 | 0.017 |
| 1/2000 | 0 | 0.012 | 0.006 | 0 | 0.009 | 0.0045 | 0.278 | 0.12 | 0.199 | 0.97 | 0.888 | 0.929 | 0 | 0 | 0 |
| 1/4000 | 0.012 | 0.005 | 0.085 | 0.016 | 0 | 0.008 | 0.061 | 0.081 | 0.071 | 0.617 | 0.505 | 0.581 | 0 | 0 | 0 |

### Example 3

### Western Blot Analysis

The specificity of clones 14C8, 6B12, and 6A12 were analyzed by western blot assay techniques. Supernatants from each of the clones bound specifically to ERB and showed no cross-reactivity with ERA protein.

Cell or tumor lysates were used for the western blots. Cell pellets or tumor samples were homogenized in a high salt buffer (20 mM Tris-HCl, pH 7.5; 2 mM DTT, 0.4 M KCl; 20% glycerol) containing a mixture of protease inhibitors (2.5 μg/ml aprotinin, antipain, leupeptin, and pepstatin plus 0.3 mM phenylmethylsulfonyl fluoride; Sigma Chemical Co., St. Louis, Mo.). Homogenates were then centrifuged at 100,000×g for 1 hour, and the supernatants stored at −20° C. until they were assayed. Homogenates (50-100 μg) were electrophoresed on 10% SDS-PAGE gels and transferred onto nylon membranes (Schleicher and Schuell, Keene, N.H.).

The blots were first stained with StainAll dye (Alpha Diagnostic International, Inc. San Antonio, Tex.) to confirm uniform transfer of all samples and then incubated in blocking solution (5% nonfat dry milk in TBST). After brief washes with TBST, the filters were then reacted with ERB monoclonal antibody at a dilution of 1:20 or 1:200 for 1 hour at room temperature, followed by extensive washes with TBST. Blots were then incubated with horseradish peroxidase-conjugated secondary antibody (Amersham Corporation, Burlington, Mass.) for 1 hour, washed with TBST, and developed using an enhanced chemiluminescence process (Amersham Corporation, Burlington, Mass.). In order to determine levels of non-specific staining, primary antibody (i.e., anti-ERB antibody) was eliminated in some experiments and secondary antibody was incubated with the samples by itself. Positive controls were run and included the use of the mammalian expression vectors (pSG5) containing the complete open reading frames of ERB1 and ERB2, which were translated in vitro in a 50 μl rabbit reticulocyte translation reaction (Promega, Madison, Wis.). Supernatants (5 μl) were analyzed by western blotting. In addition, western blots were run on homogenates of ERA- and ERB-transfected cells. For example, antibody from clone 6A12 was specifically bound in the ERB-transfected cells and did not bind to the ERA-transfected cells.

### Example 4

### Immunocytochemical Analysis

Both ERA- and ERB-transfected cells were reacted with antibody from clone 6A12 and the binding of the antibody visualized using immunoperoxidase staining techniques. The 6A12 antibody had no cross-reactivity or staining in the ERA-transfected cells, while the control ERA antibody showed nuclear staining. In contrast, in ERB-transfected cells, 6A12 antibody showed both nuclear and cytoplasmic staining while the control ERA antibody showed no cross-reactivity or staining.

Antibodies from the ERB clones were also tested to verify that they would stain fixed, paraffin-embedded tissue samples. Six samples of breast tumors, 3 tumors being ERA-positive and 3 tumors being ERA-negative, were stained with the anti-ERB antibodies. All six tumor samples stained with the anti-ERB antibody verifying that these antibodies stained archival tumor samples.

Slides were made of tumor sections cut 3-4 microns thick and baked overnight at 58° C. and deparaffinized in a Shandon-Lipshaw Varistain™, Shandon-Lipshaw Manufacturing Corporation, Pittsburgh, Pa. Slides were removed from the Varistain and transferred to a container containing 1×Tris-buffered saline.

For heat-induced antigen retrieval, all antibodies were run using 0.1 M Tris-HCl (pH 9.0). Antigen retrieval was achieved by placing the slides in an infrared hot plate/pressure cooker at 120° C. for 5 minutes. Slides were rinsed in nanopure water (six changes) and placed in 1×Tris-buffered saline. Endogenous peroxidase was blocked using a 3% hydrogen peroxide solution for 5 minutes at room temperature and then rinsing with nanopure water (three changes). Blocked tissue sections were placed in 1×Tris-buffered saline for at least 5 minutes.

The Vector avidin-biotin blocking kit (Vector Laboratories, Burlingame, Calif.) was used to block non-specific binding; this involved placing 3 drops of avidin solution A into each well formed by the slide-coverplate complex and incubating for 15 minutes, rinsing each well with 1×Tris-buffered saline, draining each well, placing 3 drops of biotin B solution into each well and incubating for 15 minutes, rinsing each well as before, and finally draining each well. Secondary antibody was then applied (biotinylated rabbit anti-mouse; 1:200 dilution; Dako, Carpinteria, Calif.) and incubated with the sections for 30 minutes. Sections were rinsed with 10×Tris-buffered saline before the application of a freshly prepared horseradish peroxidase labeled strepavidin (Dako, Carpinteria, Calif.) at a 1:200 dilution. The horseradish peroxidase labeled strepavidin was incubated with the samples for 30 minutes at room temperature. The slides were then stained with diaminobenzidine by incubating each section for 15 minutes at room temperature with three drops of diaminobenzidine solution and then rinsing each section in six changes of nanopure water, followed by transfer of the sections to a humidity chamber. After wiping the excess nanopure water from each slide, three drops of 0.2% osmium tetroxide were applied to each slide and the slides incubated for 30 seconds at room temperature. The sections were rinsed in six changes of nanopure water. All slides were counter-stained with 0.05% methyl green for 30 seconds. The stained slides were dehydrated, cleared and mounted.

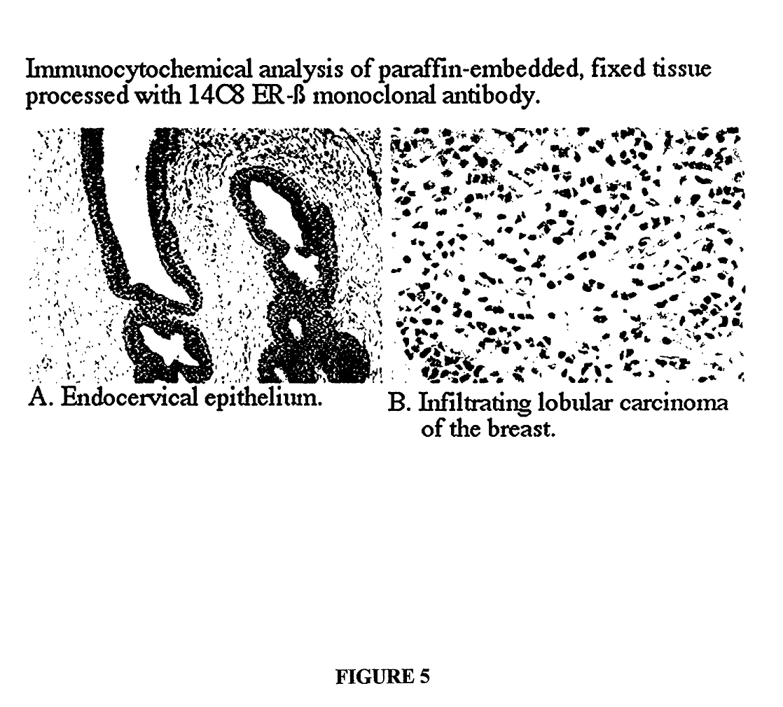

FIG. 5 is an example of an immunocytochemical analysis of paraffin-embedded fixed tissues processed with 14C8 ER-β monoclonal antibody. In FIG. 5A, endocervical epithelium tissue is treated is shown. FIG. 5B, illustrates similarly treated tissue from infiltrating lobular carcinoma of the breast.

### Example 5

### Immunocytochemical Diagnostic Kit

One embodiment of the present invention is a kit for detection of ERB in biological samples. Such a kit includes, but not limited to, a kit for immunocytochemical analysis of tissue samples. Such immunocytochemical kits are routinely used in the diagnosis and monitoring of diseases such as cancer as these techniques allow one of skill to recognize normal versus abnormal structures in individual cells and to link those abnormalities to some marker of disease, such as the presence of ERB.

The kit of the present invention would include ready-to-use monoclonal antibodies of the present invention that target the ERB. In a preferred embodiment, the kit would contain the monoclonal antibody 14C8. A kit of the present invention would include a blocking reagent of normal rabbit serum, a primary antibody (14C8), a biotinylated rabbit anti-mouse secondary antibody, avidin, biotinylated horseradish peroxidase, hydrogen peroxide solution, and diaminobenzidine tablets.

### Example 6

### Recognition of all ER-Beta Forms in Tissues

Antibodies that recognize all forms of ER-β were characterized in a manner similar to those for the forms discussed above. These antibodies were used to develop and characterize antibody reagents generated specifically against estrogen receptor beta isoforms ER-β2-5 and ER-βcx. Alterations in the amounts and/or cellular localization of ER-β and its isoforms is likely a clinically important biological feature in tumors where ER-β is expressed (e.g. breast, ovary, prostate). The analysis of ER-β and determination of the protein levels for the specific isoforms will assist the clinician to detect, treat and prevent breast, as well as other cancers (e.g. ovarian, prostate). Testing will be accomplished by:

1. Prepare the molecular reagents necessary for analysis of estrogen receptor beta (ER-β) in breast and other cancers. cDNA probes and gene specific primers using nonconserved nucleotide sequences will be used to analyze mRNA levels. Polyclonal and monoclonal antibodies will be produced that specifically recognize each isoform of ER-β. These will be used for the detection of protein levels in direct immunoblotting, immunoprecipitation, immunofluorescence and immunohistochemistry experiments.

2. Determine and correlate the protein and messenger RNA expression levels, in normal and tumor cell lines.

3. Measure levels of the ER-beta isoforms in 200 human breast tumor samples from the Baylor SPORE Breast Cancer Developmental Bank. Determine how the levels and/or cellular localization (cytoplasmic vs. nuclear) correlate with other prognostic indicators, including ER, PgR, DNA ploidy, S-phase fraction, p53, HER-2/neu, or histologic grade.

Optimal conditions will be determined using standard approaches know well to those of skill in the art of immunohistochemistry that will give adequate and specific immunostaining in frozen and archived formalin-fixed, paraffin-embedded tissue/tumor sections isolated and processed over a wide range of conditions. Tumor analysis will be extended by doing a large retrospective study of patients that received no systemic adjuvant therapy following local surgery. This type of study will provide an ideal setting for evaluating the importance of ER-β and ERB isoforms as potential prognostic factor in breast cancer.

Preliminary Data

To generate the ER-β monoclonals a fusion protein containing amino acids 1-153 was used which has very little homology to ER-α. The antibodies tested have been mapped to two regions of the ER-β protein. For the monoclonal antibodies 14C8 and 4D2 the epitope is located within the first 36 amino acids and for clones 6A12 and 6B12 the epitopes are located within amino acids 36-153.

Results of immunoblotting using in vitro translated protein indicate that none of the antibodies cross reacted with ER-α and confirm the epitope mapping results. A summary of the initial immunohistochemistry results using breast cancer tissue are shown below. These results indicate that one of the antibodies gives a strong specific nuclear signal in a ER-β positive tumor indicating that this antibody is recognizing ER-β in formalin-fixed paraffin-embedded tissue and will be a useful reagent.

| | 14C8 | 6B12 | 6A12 |
|---|---|---|---|
| WB with IVT protein | +, single band, FL | +, 2 bands, FL and a | +, 2 bands, FL and a |
| | | smaller band | smaller band |
| Permanent IHC | Good nuclear signal | Weak cytoplasmic signal | N/A |
| Conclusion | Best of any reagent at | No further testing | |
| | this point. | | |

In a comparison to other antibodies available, none gave nuclear staining in our immunohistochemical assays using formalin-fixed paraffin-embedded tissues. These include the antibodies used by Fuqua, et al. (Fuqua, S. A. W. et al. 1999. Cancer Res. 59:4525-4528) analyzing ER-β protein expression in breast cancer cell lines and tumors. While these antibodies worked well in immunoblotting and immunoprecipitations, the polyclonal antibody and monoclonal antibody both gave weak cytoplasmic staining. Commercially available antibody used in the study by Jarviene, et al., (Jarvinen, T. A. H. et al. 2000. Am J Pathology 156:29-35) (PA1313 from Affinity Bioreagents, Inc.) did not work on paraffin-embedded tissue and only gave a weak cytoplasmic stain. These results confirm previously published results and indicate the importance of generating useful reagents to between detect ER-β in clinical samples.

Experimental Design and Methods for Extending Usefulness of Reagents, Tests and Kits of Invention

To prepare the molecular reagents necessary for analysis of estrogen receptor beta in breast and other cancers, the following steps will be taken. cDNA probes and gene specific primers using nonconserved nucleotide sequences will be used to analyze mRNA levels. Specific polyclonal and monoclonal antibodies will be produced that specifically recognize each isoform of ER-Beta. These antibodies will be tested for single band specificity by direct western blots and used for the detection of protein levels in direct immunoblotting, immunoprecipitation/western blotting, immunofluorescence and immunohistochemistry experiments in normal and tumor cell lines and tissues.

In order to understand the role of estrogen receptor beta (ER-β), it will be necessary to first obtain the molecular reagents necessary to analyze ER-beta's expression and dissect ER-beta's mechanism of action. These reagents will allow determinations for each ER-β isoform if there is a direct link between alterations in mRNA levels, protein expression and possibly cellular localization in normal and cancer cell lines and tissue samples. Therefore, isoform specific DNA primers will be utilized and will be used to perform RT-PCR that will allow measurement of isoform specific mRNA levels. To measure protein levels, it will be necessary to generate a series of polyclonal and monoclonal antibodies that are specific for each ER-β isoform and give single-band specificity in a direct western blot. These reagents can then be tested and developed to provide information that can be used to profile malignant cells so that clinicians can recommend the most effective treatment regimens for their patients.

Experimental Methods

Isolation of Molecular Probes for Recombinant Protein Production and Expression

The isolation of a cDNA for each ER-β isoform will be accomplished by PCR based strategies using synthetic oligonucleotides. Poly A+ RNA will be isolated from human cell lines that have been shown to express the ER-β isoforms and RT-PCR will be performed (Asubel, F. M. et al. 1998. J. Wiley and Sons). To generate the complete coding region for each protein, it will be necessary to use the previously published sequences for each isoform, and specific synthetic oligonucleotides will be synthesized corresponding to the initiating ATG and 3′ of the termination codon of the protein (Moore, J. T. et al., 1998. Biochem BioPhys Res. Comm. 247:75-78; Fuqua, S. A. W. et al. 1999. Cancer Res. 59:4525-4528). Artificial restriction sites will be added to each oligonucleotide to allow directional in-frame cloning into a vector to allow cRNA synthesis as well as expression in prokaryotic and eukaryotic cells.

Cloning and Purification Prokarvotic Recombinant Proteins

The PCR-generated coding regions for the ER-β isoforms will be fused in-frame into the appropriate vector pET28(a-c) (Novagen, Madison, Wis.). This vector system uses the T7/lac promoter (Studier, F. W. et al. 1990. Methods Enz 185:60-89) that contains the lac operator sequence for more stringent suppression of basal expression. It also contains an amino terminal and carboxyl terminal histidine peptide sequence tag (6His-Tag) for affinity purification of the recombinant protein. The recombinant protein can be identified using a His-tag antibody. This vector also contains the T7 promoter which will be helpful in accomplishing our other specific aims since it allows in vitro synthesis of our cDNA which can be translated and labeled for in vitro binding studies (see FIG. 3). These cDNAs cloned into this vector will also be used to generate site directed point mutants that change the putative phosphorylated amino acids for each of these proteins.

Initially,the E. coli BL21 or BL21LysS bacterial strains (Studier, F. W. et al. 1990. Methods Enz 185:60-89; Smith, D. B. et al. 1988. Gene 67:31-40) will be used as well as the standard induction and isolation procedures that have been established and used successfully in the laboratory. After the induction of the recombinant fusion protein (with IPTG), examination will be made of the whole cell lysate by SDS-PAGE. If the fusion protein is expressed well, a determination will be made to see if it remains soluble or forms insoluble inclusion bodies within the bacteria. If the recombinant protein is soluble, it will be purified using nickel-agarose beads (Smith, D. B. et al. 1988. Gene 67:37-40). The recombinant protein will be eluted with imidazole, and the integrity of the protein determined by staining SDS-PAGE gels with Coomassie Brilliant Blue. The amount of protein will be quantitated by the method of Bradford (Bradford, M. M. et al. 1976. Anal biochem 72:248-254). If the protein is largely insoluble, it may be necessary to modify our induction protocol by changing temperature before/after induction, time of cell harvest after induction and IPTG concentration used for fusion protein induction. If the protein remains completely insoluble after these modifications, the protein will be denatured using urea or guanidnium hydrochloride and slowly renatured by dialysis with decreasing concentrations of the denaturant to obtain soluble protein.

Although histidine tagged recombinant proteins have provided a simple method to obtain highly purified protein, problems in level of expression and solubility of the protein may occur. If this happens, it will be necessary to use a different expression system. Since expression using other bacterial systems (pGEX, GST-fusion, etc.) may have similar problems, it may become necessary to employ the baculovirus system to express these troublesome proteins. The cDNAs will be cloned into a baculovirus expression vector pMe1Bac (A, B and C)(Invitrogen, Carlsbad, Calif.) and co-transfected with Bac-N-Blue DNA, pure recombinant plaques will be isolated, then amplified and infected into “High Five” insect cells which provide higher levels of expression than Sf9 insect cells. Alternatively, a yeast system can be used, which has the advantage of yielding proteins with mammalian-cell-like post-translational modifications.

Cloning of Eukaryotic Expression Vectors

All ER-β isoform cDNAs will be cloned into the pcDNA3.1 (Clonetech, Palo Alto, Calif.) expression vector in order to analyze antibody specificity. These vectors will also be used in future studies on receptor-receptor interaction and the effects of specific estrogenic compounds and estrogen agonists and antagonists.

Generation and Screening of Monoclonal Antibodies

Generally, polyclonal and monoclonal antibodies will be produced using, fusion proteins and synthetic peptides to generate specific epitopes to distinguish the protein isoforms. For each recombinant protein/synthetic peptide antigen, 5 six week old Balb/C female mice will be obtained from Harlan Labs and housed at the UTHSCSA animal facility (LAR-UTHSCSA; San Antonio, Tex.) will be injected. Blood from all animals that give a good immune response will be collected after the second and third boosts, the polyclonal serum pooled and used for preliminary experiments. Two animals with the highest titer of specific antibodies for monoclonal production will be utilized. If necessary a second fusion will be done using the other animals. Freund's adjuvant will be used because of its ability to stimulate a strong and prolonged immune response. The adverse side effect is the potential for invoking granuloma formation. To avoid this, injections will be made using complete Freund's adjuvant (CFA) in the primary injection and use will be made of incomplete Freund's adjuvant (IFA) for boosts. Serum will be collected to analyze the titer of serum antibodies, 5-7 days after each boost. Normally 100-200 ul of blood and will not exceed more than 300 ul. The mice will bled by nicking the vein behind the eye with a size 11 blade. The animals will be anesthetized using metafane, which will be administered before the procedure via inhalation. Using 5 animals per antigen will also allow obtaining sufficient amounts of polyclonal antibody from serum bleeds to initiate the research experiments while preparing monoclonal antibodies. All procedures will be done according to IACUC-approved protocols.

The injection protocol is listed in the table below.

| | Dose, Volume, | Route of | Frequency of | |
|---|---|---|---|---|
| Agent | Vehicle | Administration | Administration | Anticipated Effects |
| Antigen | 50 ug, 100 ul., CFA | subcutaneous (3 sites) | 1 | |
| Antigen | 50 ug, 100 ul, IFA | subcutaneous (3 sites) | 2-3x @ 3 weeks | Immune Response |
| Antigen | 50 ug, 100 ul, PBS | intravenous (tail vein) | 1 | granuloma formation |

More specifically, monoclonal antibody production using synthetic peptides that are specific for each ER-β isoform will be performed. Groups of 5 mice will be immunized with one peptide. The initial injection will be using 150 ug of purified protein or synthetic peptide that has been coupled to Keyhole Limpet Hemocyanin (KLH) or Multi-Antigenic Peptide (MAP) with complete Freunds adjuvant (CFA) per mouse subcutaneously. Two more immunizations will be performed at 2-3 week intervals, using similar amounts of antigen in incomplete Freunds adjuvant (IFA). After the second immunization and each subsequent injection, the titers for specific polyclonal antibodies will be tested by ELISA. To obtain polyclonal antibodies for initial experiments, the mice will be bled by nicking the vein behind the eye (100-200 ul) one week after each boost. After the titer for the peptide-specific antibodies has been induced to appropriate levels, the best responding mouse for each protein will be used for monoclonal antibody production. The final immunization, without adjuvant, will be by tail vein injection. Three days later the mice will be sacrificed. Spleens will be harvested and prepared for PEG-mediated cell fusion with myeloma NS-1 cells (Zhu, X. et al. 1995. J Biol Chem 270:19545-19550; Chen, Y. M. et al. 1995. Science 270:789-791 54). The fusions will be plated by limiting dilution, and the resulting hybridoma supernatants will be tested by ELISA for reactivity with the appropriate recombinant protein. The initial positive supernatants will then be propagated and retested by ELISA, with the resulting secondary-screen positive supernatants being used for immunoblotting (see below). These procedures have been used successfully by the inventors in the generation of other monoclonal antibodies such as those made against BRCA1, ER-Alpha, ER-βeta, PgR and SMRT.

After confirmation that each hybridoma is producing antibodies that show specificity by immunoblotting using the recombinant proteins produced above, they will be single-cell cloned to generate a pure population of antibody-producing cells. Identification will be made of about 10-15 positive hybridoma supernatants for each fusion that will be characterized.

The inventors have found that is critical to generate monoclonal antibodies for detection of estrogen receptor epitopes in tumor tissue samples. They have found and disclose for the first time herein that while most antibodies will work well on sections that have been fixed with organic solvents or paraformaldehyde, sometimes the epitope is hidden by a cell structure that may be unmasked with protease treatment, or the epitope is destroyed during fixation. If this occurs with one of the antibodies to an isoform that has been determined to have altered expression levels/localization (which, therefore makes it a good candidate for diagnostic use), it will be necessary to generate new antibodies specifically for use in immunohistochemistry. One possible way to generate antibodies that will not be affected by the fixation procedure is to immunize mice with paraformaldehyde-treated antigens and to screen resultant hybridomas by immunohistochemistry.

Correlation of Protein and Messenger RNA Expression Levels in Tumor/Normal Tissue

Recent evidence has shown that expression of ER-β and ER-β isoforms are expressed in normal breast epithelial cells and breast tumors and breast cancer cell lines. Using isoform specific synthetic oligonucleotide primers, measurements will be made of the mRNA levels by RT-PCR and proteins levels using the isoform specific antibodies generated above. These studies will extend the previously published work and analyze a number of different normal and cancer cell lines (breast, ovarian, prostate). Comparisons will be made of the levels of ER-β and ER-β isoforms proteins by direct western blot and/or immunoprecipitation (IP)/western blot analysis and the cellular localization by immunofluorescence and immunohistochemistry in these human cell lines.

Analysis of Estrogen Receptor-Beta Protein in Normal and Cancer Cell Lines

It has been shown previously that the mRNA levels of ER-β isoforms is cell line dependent. Approximately 20 different breast cancer cell lines (e.g., MCF-7, T47D, BT-20) and normal breast epithelial cells (e.g., HBL-100), as well as several ovarian (e.g., CAOV3, SKOV3) and prostate (e.g., DUI45) cancer cell lines will be analyzed for their expression of each ER-β isoform at the protein and mRNA level. Briefly, to analyze protein levels, 1-5×10⁶ cells will be harvested in either EBC buffer (50 mM Tris pH7.4, 120 mM NaCl, 1 mM EDTA, 1 mM 1 mM β-mercaptoethanol, 0.5% NP-40) or lysis 250 buffer (50 mM Tris pH 8.0, 250 mM NaCl, 5 mM EDTA, 50 mM NaF, 0.1% NP-40). The extracts will be incubated at 4 C. for one hour and centrifuged, and the insoluble material discarded. The protein concentration will be determined by the Bradford method, and 100-200 μg of lysate will be used. SDS/PAGE, direct western and immunoprecipitation/western blotting analysis will be performed using standard procedures. Only antibodies detecting a band of the appropriate size for the endogenous protein will be used for testing in immunofluorescence (IF) assays (see below).

Method to Analyze mRNA in Test Cell Lines

The levels of ER-β mRNA have been shown to be altered in breast cancer, and a determination will be made to if there is also a correlation of ER-β protein level with mRNA levels in the test cell lines. These experiments will also provide a secondary method of analysis for breast cancer specimens that cannot be analyzed by IP/western blotting or immunohistochemistry. Similar primers to that which have been previously used will be used here to determine ER-β isoform mRNA levels (Moore, J. T. et al. 1998. Biochem BioPhys. Res. Comm. 247:75-78; Fuqua, S. A. W. et al. 1999. Cancer Res. 59:4525-4528). Initially, semiquantitative PCR methods will be used to analyze the amounts of ER-β isoform mRNA in these cell lines. Briefly, total RNA will be extracted from cell monolayers, the concentration will be determined, and serial dilutions of each sample (50, 25, 12.5 6.25 ng) will be added to the GeneAmp PCR kit (Perkin-Elmer) that allows reverse transcription and subsequent amplification of the synthesized cDNA. Specific primer for each ER-β isoform and b-actin (internal control) will be used. Samples will be analyzed by gel electrophoresis, fluorescence quantitated, and results standardized to b-actin.

Analysis of ER-β Isoform Expression and Cellular Localization in Cancer Cell Lines

The normal breast epithelial cell lines and various cancer cell lines will also be evaluated for expression of ER-β protein isoforms using standard immunofluorescence and/or immunohistochemical (IHC) methodologies. For immunofluorescence cells will be seeded on glass cover slips and processed using fluorescent labeled secondary antibodies. For immunohistochemistry, concentrated pellets of each cell line will be processed to paraffin blocks, and histological sections from the blocks will be the substrated for the IHC assays. In these protocols, the previously produced ER-β monoclonal antibodies will be used, as well as any new isoform specific monoclonal antibodies that have been tested in immunoblotting of control and cancer cell lines. All protocols will use several dilutions to eliminate any nonspecific cross reactivity that has been previously reported as a potential problem in staining for ER-α. As positive controls for staining each cell line will also be transfected with the corresponding isoform expressing eukaryotic vector.

Immunofluorescence in Cell Lines

Since the potential exists that the monoclonal antibodies will work in a western and/or immunoprecipitation assay, but not detect the antigen in whole cells, it maybe necessary to analyze cellular localization by immunofluorescence. Cells that have been fixed using organic solvents (methanol) or paraformaldehyde will be used to determine if the monoclonal antibodies will have cross-reactivity in cell staining procedures by indirect immunofluorescence using a fluorochrome-conjugated secondary antibody. The monoclonal antibodies will be tested using both hybridoma culture supernatants and purified antibodies isolated from the supernatants. The monoclonal antibody against an 84 kDa nuclear matrix protein will be used as a positive control for nuclear staining. This protein, initially isolated by its interaction with the amino terminal region of the retinoblastoma protein, has been used extensively as a nuclear-specific marker in both immunofluorescence and cell fractionation studies (see FIG. 2 ref. 71). Specifics of the basic staining procedure for immunofluorescence are as follows:

1. Plate 8×10³ cells/well in a 24 well dish on glass coverslips (+/−poly-lysine).

2. After 20-24 hours, rinse cells briefly with PBS.

3. Fix cells either with 4% paraformaldehyde in PBS or (a) 1% formalin, (b) methanol, (c) acetone.

4. Rinse with PBS.

5. Incubate with 0.1% NP-40 in PBS (or Triton-X100).

6. Wash 2×with PBS.

7. Incubate samples in 4-10% goat serum for 1 hour at room temperature.

8. Wash with 1% BSA in PBS.

9. Incubate primary antibody for 1 hour at room temperature or overnight at 4° C.

10. Wash 3×with 1% BSA in PBS.

11. Incubate with fluorescent secondary antibody for 45 minutes at room temperature (in dark).

12. Wash extensively (at least 3×) with 1% BSA in PBS.

13. Counterstain with DAPI to visualize nuclei if desired.

14. Mount coverslips with aqueous mounting medium or 90% glycerol in PBS.

15. Examine slide using fluorescence microscope, store slides at 4° C. in dark.

This basic procedure will be used initially to test the sensitivity of the antibodies. Each antibody will have to be optimized by varying the primary antibody concentration and/or incubation time, and other parameters involving the cell preparation, fixation technique, primary and secondary antibody binding and the method of detection.

Immunohistochemistry using Paraffin Block Preparation (Tissue Culture Cells/Tissue)

Each cell line for each experimental condition will be grown to high density in one T150 tissue culture flask. When the cells are ready to harvest, they will be gently scraped from the flask bottom using a sterile rubber spatula. The free-floating cells will be washed, pelleted, resuspended in 10% neutral buffered formalin for 6 hours fixation, pelleted again, and resuspended in 100 μl of 2.5% warm agar in a microfuge tube. The agar quickly hardens into a gelatinous pellet of cells that can be processed to a paraffin block just as if it were a routine tissue biopsy. Histological sections will be cut from the blocks at 4 μm and float-mounted on adhesive (silanized) glass slides.

A standard, sensitive, generic detection system will be used with each ER-β primary antibody. This promotes both technical and cost efficiency and uniformity of results. Each antibody may require certain minor modifications for optimum performance, which usually involves adjustments of antibody concentration, incubation time, or the use of various antigen-enhancement techniques. The detection system is based on linking the primary antibodies to biotinylated secondary antibodies to peroxidase-conjugated streptavidin. The signal is developed with diaminobenzidine/hydrogen peroxide as the chromogene, which is enhanced with osmium tetroxide, and contrasted to alight methyl-green counterstain. Specifics are as follows:

Generic Detection System

1. Preparing sections

cut at 4 μm

float-mount on PLUS-coated slides in an adhesive free water bath

dry overnight at room temperature

2. Deparaffinizing

graded xylene, alcohols, and distilled water

3. Antigen retrieval (depending on primary antibody)

none, or

0.1 M citrate buffer at pH=6.0 for 5 minutes in pressure cooker at boil, or

pronase E at 1 mg/ml in PBS for 2 minutes

4. Block endogenous peroxidase

0.1% sodium azide/3% hydrogen peroxide in automation buffer for 15 minutes

5. Block non-specific protein (antibody) binding

10% chicken ovalbumin in Tris saline for 10 minutes

6. Primary antibody

2 hours to overnight (depending on antibody) at predetermined concentration in diluent buffer

7. Linking antibody (Dako, Carpinteria, Calif.)

biotinylated anti-mouse or anti-rabbit antibody at 1:100 in diluent buffer for 1 hour

8. Streptavidin-peroxidase conjugate (Dako, Carpinteria, Calif.)

1:100 in diluent buffer for 1 hour

9. Chromogene

diaminobenzidine at 1 mg/ml in automation buffer with 1 μl H₂O₂ for 30 seconds to 10 minutes

10. Signal intensification

0.2% osmium tetraoxide in PBS for 1 to 10 minutes

11. Counterstain

1% freshly prepared methyl green for 5 minutes

12. Dehydration and mounting

graded alcohols and xylene

13. Coverslip

cover section with mounting media, Permount (electron Microscopy Sciences, Ft. Washington, Pa.) and glass coverslip

For details of procedures, see Durfee, T. et al. 1994. J. Cell Biology 127:609-622; Allred, D. C. et al. 1993. J. Natl Cancer Inst 85:200-206; Oesterreich, S. et al. 1996. Clin Cancer Res 2:1199-1206; Diab, S. G. et al. 1997. Breast Cancer Res Treat 43:99-103; Makris, A. et al. 1997. Breast Cancer Res Treat 44:65-74; Berardo, M. et al. 1998. Cancer 82:1296-1302; Allred, D. C. et al. 1993. J Histotechnol 16:117-120.

Expected Results:

From these experiments, we will complete out initial analysis of ER-β monoclonal antibodies. Using western blot analysis, we will eliminate antibodies that have nonspecific cross reactivity with other cellular proteins isoforms and ER-α and would give anomalous results for cellular localization and in immunohistochemistry. The preliminary analysis using immunofluorescence and immunohistochemistry using tissue culture cell lines allows the determination to be made of the potential usefulness of this technique for analysis of tumor samples and the sensitivity and specificity of each antibody.

Measuring ER-Beta Isoforms in Archived Breast Tumor Samples

To measure levels of the ER-beta isoforms in 200 human breast tumor samples from the Baylor SPORE Breast Cancer Developmental Bank, the following steps will be taken. It will be necessary to determine how the levels and/or cellular localization (cytoplasmic vs. nuclear) correlate with other prognostic indicators, including ER, PgR, DNA ploidy, S-phase fraction, p53, HER-2/neu, or histologic grade. Currently, there are several types of markers that are used to make management and treatment decisions for patients with primary breast cancer. Prognostic factors are used to estimate the chances of disease recurrence if no systemic adjuvant therapy is given. Examples of prognostic factors are axillary lymph node status, tumor size, histologic grade, and steroid receptor status. Patients with a high estimated chance of disease recurrence more often receive systemic adjuvant therapy following local surgery. Predictive factors are used to select optimal therapies for individual patients.

Examples of predictive factors are steroid receptors, S-phase by DNA flow cytometry, and, more recently, HER-2/neu status (Allred, D. C. et al. 1998. Modern Pathology 11(2):155-168). Other newer markers such as heat shock proteins Oesterreich, S. et al. 1996. Clin Cancer Res 2:1199-1206; Elledge, R. M. et al. 1994. Cancer Research 54:3752-3757), p53 (Allred, D. C. et al. 1998. Modern Pathology 11(2):155-168), and apoptosis (Diaz-Cano, S. J. et al. 1997. Diagn. Mol. Path. 6:199-208) are not yet used routinely in the clinic, but they have provided important information about the biology of breast cancer, and their prognostic and predictive abilities are currently being evaluated.

ER-beta protein isoforms, which are involved in estrogen signal transduction and transcriptional regulation, may also be important prognostic and/or predictive markers for breast cancer and other types of cancer. To address this issue definitively, it will be necessary to correlate the level of these proteins with the clinical outcomes of patients with breast cancer. Required first steps before such definitive studies can be designed and conducted are to obtain preliminary information about the levels of these proteins that are present in human breast tumors, to determine the variability of expression of these factors among tumors, to determine how often these factors are present in the cytoplasm or the nucleus, and to determine if the levels or the localization correlate other established prognostic or predictive factors. Since no correlation has been reported between expression and tumor grade and alteration in cellular localization, differential expression of the levels and localization among tumors may be seen. Associations with other prognostic factors that are known to correlate with tumor grade (e.g., steroid receptor status and S-phase fraction) may also be seen. The magnitude and variability of the expression of the ER-β isoforms and the correlation with other factors will greatly influence sample size requirements for subsequent correlative studies.

To address these issues, IHC will be used to test the antibodies of the invention for determining the levels and cellular localization in tumor samples from the Baylor SPORE Breast Cancer Development Bank. Many other factors, including ER, PgR, DNA ploidy, S-phase faction, p53, HER-2/neu, and histogolic grade have already been measured in these tumors, but no clinical follow-up is available for any of these patients. Results of these studies will be used to determine which proteins will be best for further study. These results will also be used to refine the sample size requirements for the follow-up study.

Methods

Immunohistochemistry and Analysis of Protein Levels

The levels of ER-βeta isoform specific antibodies will be determined by IHC using procedures previously described. ER levels and PgR levels have been previously determined by biochemical assays (Dressler, L. G. et al. 1988. Cancer 61:420-427) and by IHC using the 6F11 antibody from Novocastra for ER (Harvey, J. M. et al. J Clin Oncol (in press)) and the KD68 antibody from Abbott Laboratory for PgR (6156); DNA ploidy and S-phase fraction have been previously determined by flow cytometry. (Dressier, L. G. et al. 1988. Cancer 61:420-427, Wenger, C. R. et al. 1993. Breast Cancer Res Treat 28:9-20); p53 has been measured by IHC using a cocktail of antibodies that includes Pab 1801 and Pab 240 from GeneTex (Allred, D. C. et al. 1993. J Natl Cancer Inst 85:200-206), and HER-2/neu has been previously measured by IHC using the TAB 250 antibody from Triton (Elledge, R. M. et al. 1998. Clin Cancer Res 4:7-12). Immunohistochemistry procedures will be as previously described.

In addition to the information and expression levels detected using IRC, the levels of protein will be determined by western blotting an/or immunoprecipitation/western blotting techniques. In these procedures pulverized samples of the identical tumor that was used in IHC will be lysed in 5% SDS, and the protein concentration will be quantitated using BCA reagent assay (Pierce, Rockford, Ill.). These levels can then be correlated to the scoring of the IHC and to the expression of the other predictive markers that have previously been documented from these tumor samples.

Statistical Analyses

Each protein's levels will first be analyzed as continuous variables. For prognostic factors that are measured on a continuous scale (ER, PgR, S-phase fraction, p53, HER-2/neu), associations with each protein will be assessed by Spearman non-parametric correlation coefficients. For factors that are inherently dichotomous or are usually classified into two categories (DNA ploidy, ER status, PgR status, p53 status, HER-2/neu status), Wilcoxon non-parametric rank sum tests will be used to compare protein levels. The distributions of IHC scores for each protein will then be examined to see if there any natural breakpoints that might be used to differentiate low levels from high levels, and appropriate procedures for binary variables (Chi-square tests, Fisher exact tests) will be applied. With a sample of 200 specimens, the width of a 95% confidence interval for rate of over-expression of each protein will be less than 14%. If the data follow normal distributions or can be transformed to normal distributions, an 80% power to detect a correlation of 0.195 and 90% to detect a correlation of 0.224 with any of the standard prognostic factors is predicted.

What is claimed is:

1. A monoclonal antibody said monoclonal antibody having binding specificity for a single epitope of the estrogen receptor-β protein.

2. A monoclonal antibody said monoclonal antibody having binding specificity for a single epitope of the estrogen receptor-β protein in a histological sample.

3. The monoclonal antibody of claim 2 wherein said histological sample is a fixed paraffin-embedded tissue.

4. The monoclonal antibody of claim 3 wherein said tissue is selected of ERB-containing tissues selected from the group of breast, prostate, testis, ovary, thymus, spleen, adipose, uterus, pituitary, and kidney.

5. The monoclonal antibody of either of claims 1 or 2 wherein said epitope lies within amino acid residues 1-146 of ERB.

6. The antibody of claim 5, said antibody consisting of anti-estrogen receptor-β antibody produced by hybridoma clone 14C8.

7. The monoclonal antibody of either of claims 1 or 2 wherein said epitope lies within amino acid residues 1-36 of ERB.

8. The monoclonal antibody of either of claim 1 or claim 2 wherein said epitope lies within amino acid residues 36-146.

9. The monoclonal antibody of either of claim 1 or claim 2 where in said estrogen receptors protein is an isoform selected from the group ERβ1, ERβ2, ERβ3, ERβ4, ERβ5, and Erβcx.

10. A mono clonal antibody said monoclonal antibody having binding specificity for a single epitope of the estrogen receptor-β protein, wherein said epitope is selected from the group of epitopes lying within the amino acid ranges 1-146, 1-36 and 36-146.

11. A monoclonal antibody said monoclonal antibody having binding specificity for a single epitope of the estrogen receptor-β protein in a histological sample, wherein said histological sample is a fixed paraffin-embedded tissue.

12. A monoclonal antibody said monoclonal antibody having binding specificity for a single epitope of the estrogen receptor-β protein in a histological sample, wherein said histological sample is a fixed paraffin-embedded tissue, wherein said tissue is breast tissue.

13. A monoclonal antibody said monoclonal antibody having binding specificity for a single epitope of the estrogen receptor-β protein in a histological sample, wherein said histological sample is a fixed paraffin-embedded tissue, wherein said tissue is breast tissue, wherein said epitope is one lying within the amino acid range 1-146 of said protein, and wherein said monoclonal antibody is produced by hybridoma clone 14C8.

14. The monoclonal antibody of either of claim 1 or claim 2 selected from the group consisting of an anti-estrogen receptor-β antibody from hybridoma clones 14C8, 14G2, 4D2, 6B 12, and 6A12.

15. A monoclonal antibody selected from the group consisting of anti-estrogen receptor-β antibodies from hybridoma clones 14C8, 14G2, 4D2, 6B 12, and 6A12.

16. A hybridoma which produces a monoclonal antibody that specifically binds to estrogen receptor-β, said hybridoma comprising:
an antibody producing cell producing a monoclonal antibody having binding specificity for a single epitope on estrogen receptor-β protein; and
a tumor cell fused with said antibody producing cell.

17. A method for detection of estrogen receptor-β in a biological sample comprising:
contacting said sample with the antibody of claim 1; and
detecting the presence of estrogen receptor-β in said sample using an analytical technique selected from the group consisting of radioimmunoassay, immunoprecipitation, western blotting, enzyme-linked immunosorbent assays (ELISA), and immunocytochemistry.

18. A method for detection of a range of estrogen receptor-β isoforms in a biological sample comprising:
differentially labeling at least two antibodies of claim 9, each antibody specifically and uniquely recognizing one of said isoforms
contacting said sample with the antibodies; and
detecting the presence of estrogen receptor-β isoforms in said sample using an analytical technique selected from the group consisting of radioimmunoassay, immunoprecipitation, Western blotting, enzyme-linked immunosorbent assays (ELISA), and immunocytochemistry.

19. A kit for detection of estrogen receptors in a biological sample comprising:
a blocking reagent;
a monoclonal antibody of either of claim 1, claim 2 or claim 9;
a secondary antibody;
an avidin solution;
a biotinylated horseradish peroxidase solution;
a hydrogen peroxide solution; and
diaminobenzidine tablets.
